(19) United States
(12) Patent Application Publication
(10) Pub. No.: US 2009/0306209 A1
(43) Pub. Date: Dec. 10, 2009

(54) ENTERIC-COATED FORMULATIONS OF POLYETHYLENE GLYCOL AND ONE OR MORE SOLUBLE AMINO ACIDS FOR ORAL INGESTION AND ENHANCED UPTAKE OF SAME

(76) Inventors: F. Joseph DAUGHERTY, Omaha, NE (US); Michael S. TEMPESTA, El Granada, CA (US)

Correspondence Address: HOGAN & HARTSON LLP, ONE TABOR CENTER, SUITE 1500, 1200 SEVENTEENTH ST, DENVER, CO, 80202, US

(21) Appl. No.: 12/477,724

(22) Filed: Jun. 3, 2009

Related U.S. Application Data
(60) Provisional application No. 61/058,838, filed on Jun. 4, 2008.

(51) Int. Cl. A61K 31/197; A61P 7/06

(52) U.S. Cl. 514/563

(57) ABSTRACT

Oral amino acid formulations comprising polyethylene glycol are enteric coated. Most preferred amino acids are leucine, glutamine, and arginine. The most preferred polyethylene glycols have an average molecular weight of from 3150 to 3685, although for particular formulation formulations and particular uses, the average molecular weight polyethylene glycols may range from 190 to 9000.

### RELATED APPLICATION

The present application claims priority of U.S. Provisional Patent Application Ser. No. 61/058,838, which is incorporated herein in its entirety by this reference.

### FIELD OF THE INVENTION

The present invention relates to leucine, glutamine and arginine formulations adapted for use as a source of oral leucine, glutamine and arginine. The present invention further relates to formulations containing a combination of one or more amino acids adapted for increased uptake in an oral form.

### BACKGROUND OF THE INVENTION

The oral ingestion of leucine, glutamine, arginine and other amino acids in the form of a supplement is known. Indeed, such oral supplementation is broadly used as a performance enhancement practice by athletes.

However, although leucine, glutamine and arginine are stable ex vivo, certain forms of these amino acids have suboptimal bioavailability and are unstable in vivo, i.e., in the acidic environment that exists in the stomach, and the basic conditions of the lower gastrointestinal tract. The stability problem may be further exacerbated when the leucine, glutamine or arginine is dissolved in fruit juices and other acidic liquids, which may promote degradation.

In particular with respect to glutamine, it is known that glutamine has a role in protein synthesis, especially with respect to muscle growth, as glutamine is said to be responsible for 35% of the nitrogen transported into the muscle cell. Glutamine also has important anti-proteolysis effect, preventing muscle tissue breakdown. However, after heavy exercise, glutamine levels fall and for this and other reasons, numerous supplements containing glutamine are commercially available. For one such product, a recommended serving dose is 10 g per day of L-glutamine. It is recommended that the powdered product be mixed with water or juice.

Glutamine has been studied extensively over the past 15 years and has been shown to be useful in treatment of serious illnesses, injury, trauma, burns, cancer, side effects of cancer treatment, and would healing. In catabolic states of injury and illness, glutamine because conditionally essential, requiring intake from food or supplements. Enhanced glutamine uptake may be achieved when taken as a tablet in suppository form, as most amino acids are absorbed in the lower stomach. It has also been found that glutamine can reduce healing time after surgery, with hospital stay times after abdominal surgery reduced by providing parenteral nutrition containing glutamine.

Other biochemical functions of glutamine include acting as a substrate for DNA synthesis, constituting a primary source of fuel for the cells lining the inside of the small intestine, helping to block cortisol-induced protein catabolism, acting as a carrier of ammonia from extra-hepatic tissues, contributes to the regulation of the acid-base balance in the kidneys, and acts as a precursor for rapidly dividing immune cells, thereby aiding in the immune function. Other reported uses include treatment of anxiety and depression, use as an anti-inflammatory in the treatment of autoimmune diseases, and use to assist diabetics in the management of sugar cravings.

Glutamine also is reported to have an effect on brain function, with glutamine is said to provide an alternative source of fuel for the brain, as well as enhance brain function by fueling glutamic acid and gamma-aminobutyric acid-two of the brain's important neurotransmitters. Glutamine also has been used to aid memory, as it assists in nitrogen transportation and reduces toxic buildup of ammonia in the brain (although contra-indicated for those with Reye's Syndrome).

L-glutamine therapies have been found to be relevant to treating sickle cell anemia. See, e.g., U.S. Pat. No. 5,693,671. In one 2005 publication it was reported that oral L-glutamine administration consistently resulted in improvement of sickle red blood cell adhesion to human umbilical vein endothelial cells. More recently, phase II clinical trials are schedule for a treatment purported to reduce the number of blood cells deformed by sickle cell anemia.

Arginine is also taken as a supplement. Modes of action are believed to include removal from the body of ammonia, which is a waste product of protein metabolism. Arginine is also an essential precursor to nitric oxide, which helps maintain blood vessel tone. Suggested dosages range from 1 to 5 grams per day.

Furthermore, oral ingestion of arginine has been reported to stimulate release of growth hormone, improve immune function, reduce healing time of injuries, speed repair of damaged tissue, reduce risk of heart disease, increase muscle mass, improve insulin sensitivity. Some of these effects may be related to arginine being a precursor of nitric oxide, urea, omithine and agmatine and needed in the synthesis of creatine.

Leucine is an α-amino acid which has been found to slow muscle tissue degradation by increasing synthesis of muscle proteins. The term the term leucine as used herein to encompass all food-grade, physiologically safe isomers of leucine. Leucine is expected to be helpful in minimizing loss of muscle mass in the elderly, and plays a key role in the regulation of muscle protein synthesis. It helps to reduce the amount of protein breakdown from exercise and helps to preserve muscle glycogen stores. In addition, leucine is utilized in the liver and in adipose tissue in the formation of sterols. Suggested daily dosages for commercially available products are from 3 to 6.2 grams per day. In one case, a 3 gram dose requires ingestion of 6 capsules.

Accordingly, development of formulation in which the leucine, glutamine, arginine and/or other amino acids, alone or in combination, are in a form which is resistant to degradation, i.e., is stable in acid and base environments of the stomach and gut, but which is ultimately absorbed with enhanced efficacy, remains needed.

### SUMMARY OF THE INVENTION

The most preferred embodiments of the amino acid formulations of the present invention are those which include leucine, glutamine and/or arginine formulations in the form of a soluble leucine, soluble glutamine an/or soluble arginine, mixed with polyethylene glycol (PEG), after which the mixture is coated with an enteric coating. While the average molecular weight of the PEG component can vary, and indeed, there may be substantial variation in the range of PEG chain length, a preferred range of the average molecular weight of a PEG component suitable for human consumption is from 150 to 9000. A more preferable range of average molecular weight of the PEG component is from 3015 to 4800. The most preferred PEGs have an average molecular weight of from 3150 to 3685.

Preferred ratios by weight percent of leucine, glutamine or arginine equivalents to PEG are from 99:1 to 50:50. A more preferred range is from 95:5 to 90:10.

The mixture or dispersion of the PEG and the leucine, glutamine, arginine and/or other amino acid is preferably then enteric coated with an enteric coating preferably comprising cellulose, sodium alginate, medium chain triglycerides and oleic and stearic acid, and then formed into tablets. Other enteric coatings known to those of skill in the art may also be used and other delivery forms may also be made. Also, although less preferably, the amino acid component(s) and the PEG may be separately enteric coated, and then mixed for presentation in a form suitable for an oral dose, such as a tablet, gel cap, other encapsulated form, etc.

A preferred daily dosage of a glutamine equivalent of the present formulations is from 0.1 to 9 grams per day. Alternate dosages to be suitable for maintenance dosages to maintain previous loading regimens, are from 0.5 to 2 grams per day of the glutamine equivalent component of the formulation. Even lower dosages of from 0.01 to 0.5 grams per day of the glutamine equivalent of the formulation are expected to have beneficial results, especially for older people. Therapeutic does for disease conditions or those undergoing extreme exercise-related stress exceed these levels and be has high as 10-20 grams per day.

A preferred daily dosage of a leucine equivalent of the present formulations is from 0.1 to 2.5 grams per day. Alternate dosages to be suitable for maintenance dosages to maintain previous loading regimens, are from 0.05 to 1 grams per day of the leucine equivalent component of the formulation. Even lower dosages of from 0.01 to 0.5 grams per day of the leucine equivalent of the formulation are expected to have beneficial results, especially for older people. Therapeutic does for disease conditions or those undergoing extreme exercise-related stress exceed these levels and be has high as 5-10 grams per day.

A preferred daily dosage of an arginine equivalent of the present formulations is from 0.1 to 0.9 grams per day. Alternate dosages to be suitable for maintenance dosages to maintain previous loading regimens, are from 0.05 to 0.9 grams per day of the arginine equivalent component of the formulation. Even lower dosages of from 0.01 to 0.1 grams per day of the arginine equivalent of the formulation are expected to have beneficial results, especially for older people. Therapeutic doses for disease conditions or those undergoing extreme exercise-related stress exceed these levels and be has high as 1-5 grams per day.

### DETAILED DESCRIPTION OF PREFERRED EMBODIMENTS

A leucine, glutamine or arginine formulation of the present invention includes a soluble leucine, glutamine or arginine and polyethylene glycol (PEG), coated with an enteric coating. The most preferred form of the PEG component contains an average molecular weight of 3015 to 3685 and is marketed under the Carbowax® PEG 3350 trade name, which at ambient temperature, is a hard, opaque white, granular solid. The most preferred form of the soluble leucine, glutamine or arginine is a salt which at room temperature, is preferably obtained in powdered or crystalline form. The most preferred formulation of the present invention, is a solid dispersion of a leucine, glutamine or arginine salt in the PEG 3350, with the dispersion then coated with an enteric coating. Forms of leucine, glutamine or arginine acceptable for use in the formulations of the present invention are soluble leucine, glutamine or arginine defined here as those forms of leucine, glutamine or arginine of food grade quality which are soluble in room temperature aqueous solutions by weight from 2.5% to 50% within one hour with stirring.

Other preferred PEGs are opaque granular solids, including PEG having an average molecular weight of from 1305 to 1595 (e.g., Carbowax® PEG 1450), of from 3600 to 4400 (e.g., Carbowax® PEG 4000), of from 4400 to 4800 (e.g., Carbowax® PEG 4600), and of from 7000 to 9000 (e.g., Carbowax® PEG 8000). PEG having an average molecular weight of from 6000 to 7500 (e.g., Carbowax® PEG 6000) is also preferred.

A preferred daily dosage of a glutamine equivalent of the present formulations is from 0.1 to 9 grams per day. Alternate dosages to be suitable for maintenance dosages to maintain previous loading regimens, are from 0.5 to 2 grams per day of the glutamine equivalent component of the formulation. Even lower dosages of from 0.01 to 0.5 grams per day of the glutamine equivalent of the formulation are expected to have beneficial results, especially for older people. Therapeutic does for disease conditions or those undergoing extreme exercise-related stress exceed these levels and may be as high as 10 to 20 grams per day.

The most preferred form of glutamine for use in the formulations and methods of the present invention is L-glutamine which has a solubility in water at 30° C. of 5 grams/100 ml. Although L-glutamine-HCl is very soluble in water and may be used in the present invention, it is presently not commercially available. L-glutamine ester (either neutral or as an HCl salt) may be used but is also presently not readily available.

A preferred daily dosage of a leucine equivalent of the present formulations is from 0.1 to 2.5 grams per day. Alternate dosages to be suitable for maintenance dosages to maintain previous loading regimens, are from 0.05 to 1 grams per day of the leucine equivalent component of the formulation. Even lower dosages of from 0.01 to 0.5 grams per day of the leucine equivalent of the formulation are expected to have beneficial results, especially for older people. Therapeutic doses for disease conditions or those undergoing extreme exercise-related stress exceed these levels and may be as high as 5 to 10 grams per day.

The most preferred form of leucine is L-leucine, having a solubility in water at 25° C. of 2.4 g/100 ml. Also acceptable is L-leucine.HCl, which is also very soluble in water. L-leucine.ester (both as a neutral or an HCl salt) is less preferred but acceptable.

A preferred daily dosage of an arginine equivalent of the present formulations is from 0.1 to 0.9 grams per day. Alternate dosages to be suitable for maintenance dosages to maintain previous loading regimens, are from 0.05 to 0.9 grams per day of the arginine equivalent component of the formulation. Even lower dosages of from 0.01 to 0.1 grams per day of the arginine equivalent of the formulation are expected to have beneficial results, especially for older people. Therapeutic does for disease conditions or those undergoing extreme exercise-related stress exceed these levels and may be as high as 1-5 grams per day. A most preferred range of daily dosage is from 0.1 to 20 grams per day of arginine equivalent component of the formulations of the present invention.

The most preferred form of arginine.2(H₂O) which is soluble in water at 21° C. at 15 g/100 ml. L-arginine.HCl is also acceptable and is very soluble in water. L-arginine.glutamate is also acceptable and is soluble in room temperature water at 13.5 g. equivalents of arginine/100 ml.

It is expected that less total leucine, glutamine or arginine equivalent will need to be ingested to obtain the same or greater muscle uptake of leucine, glutamine or arginine, as compared to other oral formulations containing leucine, glutamine or arginine which do not contain PEG and are not enteric coated. If this is so, it is postulated that the PEG in the formulation may clear more slowly from the gastrointestinal tract, and thus be made available to the individual over a more extended period of time, possibly contributing to enhanced muscle uptake. Accordingly, it is postulated that the lower dosages of leucine, glutamine or arginine may be ingested using the compositions of the present invention, while maintaining optimal loading kinetics.

The enteric-coated, PEG and soluble leucine, glutamine or arginine blends of the present invention, and in particular the most preferred embodiment which comprises an enteric-coated dispersion of a leucine, glutamine or arginine salt dispersed in PEG is expected to be useful for minimizing symptoms of certain diseases. This same preferred form of an enteric-coated, PEG/amino acid dispersion is also preferred for use with other soluble amino acids.

In alternate embodiments, liquid formulations in a liquid PEG such as having average molecular weights of from 190 to 210 (e.g., Carbowax® PEG 200), from 285 to 315 (e.g., Carbowax® PEG 300), from 380 to 420 (e.g., Carbowax® PEG 400) and from 570-630 (e.g., Carbowax® PEG 600) may be used.

Also, in addition to the enteric-coated pill form of the leucine, glutamine, and/or arginine formulations of the present invention, other conventional enteric-coatings are contemplated, as are other forms, such as caplets and other dosing formulations. Moreover, the amino acid component(s) and the polyethylene glycol component may be mixed together first and then the dispersion enteric coated or may be separately enteric coated and then mixed in desired ratios.

Other formulations and other embodiments of the present invention are contemplated in which other soluble amino acids are mixed with PEG and then enteric coated, as described above. Such a formulation comprises a soluble amino acid selected from the group consisting of soluble alanine, soluble asparagine, soluble aspartic acid, soluble cysteine, soluble cystine, soluble glutamic acid, soluble glycine, soluble histidine, soluble isoglutamine, soluble isoleucine, soluble lysine, soluble methionine, soluble norleucine, soluble norvaline, soluble omithine, soluble phenylalanine, soluble proline, soluble pyroglutamic acid, soluble serine, soluble threonine, soluble tryptophan, soluble tyrosine, and soluble valine; and polyethylene glycol, wherein the soluble amino acid and polyethylene glycol are coated with an enteric coating.

Daily dosage ranges for the following amino acids are preferable, when used in the formulations of the present invention: L-histidine—from 0.1 to 7.5 g/day; L-tryptophan—from 0.1 to 7.5 g/day; L-phenylalanine—from 0.2 to 12 g/day; L-lycine—from 0.2 to 15 g/day; L-threonine—from 0.1 to 7.5 g/day; L-methionine—from 0.2 to 15 g/day; L-isoleucine—from 0.5 to 12 g/day; and L-valine—from 0.5 to 15 g/day.

Yet other formulations and other embodiments of the present invention are contemplated in which α-amino acids are mixed with PEG and then enteric coated, as described above.

Additional formulations are contemplated in which other compounds selected from the group citrulline, camosine, anserine, cystathionine, homocysteine, hydroxylysine and hydroxyproline, methyl histidines, sarcosine, taurine and phosphoserine are mixed with PEG, and the mixture is enteric-coated.

Most preferably, an oral formulation of the present invention comprises soluble glutamine; and polyethylene glycol, wherein the soluble glutamine and polyethylene glycol are coated with an enteric coating. The polyethylene glycol may have an average molecular weight of from 3150 to 3685, from 3600 to 4400 or from 4400 to 4800. Such oral formulations preferably are taken in a total dosage of from 0.1 to 9 grams per day of glutamine equivalents, with a total dosage of from 0.01 to 0.5 grams per day of glutamine equivalents also acceptable. Preferred usage of the oral formulations of glutamine of the present invention is to relieve, minimize or prevent symptoms of a muscle wasting disease.

While a preferred method of using the aforementioned oral formulations of glutamine is to supplement a mammal's glutamine stores by providing the formulations for ingestion by the mammal, the compositions may also be used as a supplement to build protein, increase muscle mass and supplement glutamine stores in fish and poultry.

Most preferably, an oral formulation of the present invention which includes soluble arginine also include polyethylene glycol, with the soluble arginine and polyethylene glycol coated with an enteric coating. The polyethylene glycol may have an average molecular weight of from 3150 to 3685, from 3600 to 4400 or from 4400 to 4800. Such oral formulations preferably are taken in a total dosage of from 0.1 to 0.9 grams per day of arginine equivalents, with a total dosage of from 0.01 to 0.1 grams per day of arginine equivalents also acceptable. Preferred usage of the oral formulations of arginine of the present invention is to relieve, minimize or prevent symptoms of a muscle wasting disease.

Also preferably, an oral formulation of the present invention which includes soluble leucine also include polyethylene glycol, with the soluble leucine and polyethylene glycol coated with an enteric coating. The polyethylene glycol may have an average molecular weight of from 3150 to 3685, from 3600 to 4400 or from 4400 to 4800. Such oral formulations preferably are taken in a total dosage of from 0.1 to 2.5 grams per day of leucine equivalents, with a total dosage of from 5 to 10 grams per day of leucine equivalents also acceptable. Preferred usage of the oral formulations of leucine of the present invention is to relieve, minimize or prevent symptoms of Parkinson's disease.

In yet other embodiments of the present invention, an oral formulation comprises a soluble amino acid selected from the group consisting of soluble alanine, soluble asparagine, soluble aspartic acid, soluble cysteine, soluble cystine, soluble glutamic acid, soluble glycine, soluble histidine, soluble isoglutamine, soluble isoleucine, soluble lysine, soluble methionine, soluble norleucine, soluble norvaline, soluble ornithine, soluble phenylalanine, soluble proline, soluble pyroglutamic acid, soluble serine, soluble threonine, soluble tryptophan, soluble tyrosine, and soluble valine; and polyethylene glycol, wherein the soluble amino acid and polyethylene glycol are coated with an enteric coating. In such oral formulations, most preferably the polyethylene glycol has an average molecular weight of from 3150 to 3685, although the polyethylene glycol may also have an average molecular weight of from 3600 to 4400, with formulations containing polyethylene glycol having an average molecular weight of from 4400 to 4800 also acceptable. These formulations preferably contain from 0.1 to 10 grams per day of amino acid equivalents.

The invention also comprehends a method of supplementing a mammal's amino acid stores by providing a mixture of polyethylene glycol and a soluble amino acid selected from the group consisting of soluble alanine, soluble asparagine, soluble aspartic acid, soluble cysteine, soluble cystine, soluble glutamic acid, soluble glycine, soluble histidine, soluble isoglutamine, soluble isoleucine, soluble lysine, soluble methionine, soluble norleucine, soluble norvaline, soluble ornithine, soluble phenylalanine, soluble proline, soluble pyroglutamic acid, soluble serine, soluble threonine, soluble tryptophan, soluble tyrosine, and soluble valine, wherein the mixture is coated with an enteric coating, and providing the enteric-coated mixture to the mammal for ingesting. Alternatively, the formulation, if biocompatible, may be used as a supplement for poultry and/or fish.

More generally, the present invention contemplates oral formulations comprising one or more soluble amino acids, preferably a-amino acids, and polyethylene glycol, wherein the one or more soluble amino acid and polyethylene glycol are coated with an enteric coating.

Other formulations include one or more compounds selected from the group consisting of citrulline, camosine, anserine, cystathionine, homocysteine, hydroxylysine, hydroxyproline, methyl histidines, sarcosine, taurine and phosphoserine, together with polyethylene glycol, wherein the compound and polyethylene glycol are coated with an enteric coating.

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be resorted to falling within the scope of the invention as defined by the claims that follow.

In particular, it is hereby noted that the alternate formulations in which the PEG is separately enteric coated, etc., as described above are also applicable to all formulations and methods described herein relating to other amino acids and molecules. Moreover, as would be understood by those of skill in the art, the term “soluble” as used above with respect to the formulations of the present invention including glutamine, leucine and/or arginine, is applicable to the other amino acids and molecules identified herein.

Finally, the words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

1. A oral formulation comprising:
one or more amino acids selected from the group consisting of soluble glutamine, soluble leucine and soluble arginine; and
polyethylene glycol,
wherein the one or more soluble amino acids and polyethylene glycol are coated with an enteric coating.

2. The oral formulation of claim 1, wherein the polyethylene glycol has an average molecular weight of from 190 to 9000.

3. The oral formulation of claim 1, wherein the polyethylene glycol has an average molecular weight of from 3150 to 3685.

4. The oral formulation of claim 1, wherein the polyethylene glycol has an average molecular weight of from 3600 to 4400.

5. The oral formulation of claim 1, wherein the polyethylene glycol has an average molecular weight of from 4400 to 4800

6. The oral formulation of claim 1, wherein a daily dosage of the formulation is from 0.1 to 9 grams per day of glutamine equivalents.

7. The oral formulation of claim 1, wherein a dosage of the formulation is from 0.01 to 0.5 grams per day of glutamine equivalents.

8. The oral formulation of claim 1, wherein a dosage of the formulation is from 0.1 to 9 grams per day of arginine equivalents.

9. The oral formulation of claim 1, wherein a dosage of the formulation is from 0.1 to 20 grams per day of arginine equivalents.

10. The oral formulation of claim 1, wherein a dosage of the formulation is from 0.01 to 0.1 grams per day of arginine equivalents.

11. The oral formulation of claim 1, wherein a dosage of the formulation is from 0.1 to 2.5 grams per day of leucine equivalents.

12. The oral formulation of claim 1, wherein a dosage of the formulation is from 5 to 10 grams per day of leucine equivalents.

13. The oral formulation of claim 1, wherein a dosage of the formulation is from 2.5 to 5 grams per day of leucine equivalents.

14. The oral formulation of claim 1, wherein the formulation after ingestion, is effective to relieve, minimize or prevent symptoms of a muscle wasting disease.

15. The oral formulation of claim 1, wherein the formulation after ingestion, is effective to relieve, minimize or prevent symptoms of Parkinson's disease.

16. The oral formulation of claim 1, wherein the one or more amino acid comprises L-glutamine and the formulation, after ingestion by a human, is effective to treat sickle cell anemia.

17. The oral formulation of claim 1, wherein the group of amino acids from which the one or more amino acids are selected further comprises soluble alanine, soluble asparagine, soluble aspartic acid, soluble cysteine, soluble cystine, soluble glutamic acid, soluble glycine, soluble histidine, soluble isoglutamine, soluble isoleucine, soluble lysine, soluble methionine, soluble norleucine, soluble norvaline, soluble ornithine, soluble phenylalanine, soluble proline, soluble pyroglutamic acid, soluble serine, soluble threonine, soluble tryptophan, soluble tyrosine, and soluble valine.

18. The oral formulation of claim 1, wherein the group of amino acids further comprises one or more soluble a-amino acids.

19. A method of supplementing an animal's uptake of one or more amino acids comprising providing a mixture of polyethylene glycol and one or more soluble amino acids selected from the group consisting of soluble glutamine, soluble leucine and soluble arginine, wherein the mixture is coated with an enteric coating, and providing the coated mixture to the mammal for ingesting.

20. The method of claim 19, wherein the group of amino acids from which the one or more amino acids are selected further comprises soluble alanine, soluble asparagine, soluble aspartic acid, soluble cysteine, soluble cystine, soluble glutamic acid, soluble glycine, soluble histidine, soluble isoglutamine, soluble isoleucine, soluble lysine, soluble methionine, soluble norleucine, soluble norvaline, soluble ornithine, soluble phenylalanine, soluble proline, soluble pyroglutamic acid, soluble serine, soluble threonine, soluble tryptophan, soluble tyrosine, and soluble valine

21. The method of claim 19, wherein the polyethylene glycol has an average molecular weight of from 3150 to 3685.

22. The method of claim 19, wherein the polyethylene glycol has an average molecular weight of from 190 to 9000.

22. The method of claim 17, wherein a daily dosage of the formulation is from 0.1 to 10 grams per day of amino acid equivalents.

23. A method of treating sickle cell anemia in a patient comprising providing efficacious enteric coated dosages of polyethylene glycol and L-glutamine for ingestion by the patient, and ingesting the enteric coated dosages by the patient.
